(19) United States
(12) Patent Application Publication
(10) Pub. No.: US 2006/0246060 A1
(43) Pub. Date: Nov. 2, 2006

(54) Novel stable formulation

(76) Inventor: Douglas P. Nesta, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/519,033

(22) PCT Filed: Jul. 2, 2003
(86) PCT No.: PCT/US03/20751

§ 371 Date: Dec. 22, 2004

Related U.S. Application Data
(60) Provisional application No. 60/393,189, filed on Jul. 2, 2002.

(51) Int. Cl. A61K 39/395

(52) U.S. Cl. 424/141.1

(57) ABSTRACT

Invented are non-peptide TPO mimetics. Also invented is a method of treating thrombocytopenia, in a mammal, including a human, in need thereof which comprises administering to such mammal an effective amount of a selected hydroxy-1-azobenzene derivative.

### FIELD OF THE INVENTION

The present invention relates to a stable formulation for huC242-DM1, an antibody conjugated to cytotoxic agent.

### BACKGROUND OF THE INVENTION

In the past ten years, advances in biotechnology have made it possible to produce a variety of proteins for pharmaceutical applications using recombinant DNA techniques. Because proteins are larger and more complex than traditional organic and inorganic drugs (i.e. possessing multiple functional groups in addition to complex three-dimensional structures), the formulation of such proteins poses special problems. For a protein to remain biologically active, a formulation must preserve intact the conformational integrity of at least a core sequence of the protein's amino acids while at the same time protecting the protein's multiple functional groups from degradation. Degradation pathways for proteins can involve chemical instability (i.e. any process which involves modification of the protein by bond formation or cleavage resulting in a new chemical entity) or physical instability (i.e. changes in the higher order structure of the protein). Chemical instability can result from deamidation, racemization, hydrolysis, oxidation, beta elimination or disulfide exchange. Physical instability can result from denaturation, aggregation, precipitation or adsorption, for example. The three most common protein degradation pathways are protein aggregation, deamidation and oxidation. Cleland et al Critical Reviews in Therapeutic Drug Carrier Systems 10(4): 307-377 (1993).

Included in the proteins used for pharmaceutical applications are antibodies. An example of an antibody useful for therapy is a murine antibody C242. See. EP 528,527B1. huC242-DM1 is a tumor-activated immunotoxin under development by GlaxoSmithKline plc as a treatment for antigen-expressing tumor types (lead indication pancreatic or PMP cancer). It consists of a humanized antibody of C242, huC242, conjugated to DM1, a new derivative of maytansinoid. There have been many reports on both C242-DM1 and huC242-DM1. See for example, Proc. Natl. Acad. Sci. USA, Vol. 93, pp 8618-8623, 1996; Current Opinion in Molecular Therapeutics 3(2):198-203, 2001.

### SUMMARY OF THE INVENTION

Accordingly, the invention provides a stable aqueous pharmaceutical formulation of huC242-DM1 (the immunoconjugate) comprising the immunoconjugate concentration range -1-20 mg/mL) in a buffer maintaining the pH in the range of ˜5.8-6.2 (50 mM succinic acid, pH 6.0), and containing sucrose (˜5% w/v); this formulation is suitable for subsequent lyophilization to create a stable dosage form.

Further provided is a stable frozen formulation for monoclonal antibody C242, comprised of the monoclonal antibody protein (concentration range ˜1-30 mg/mL) in a buffer maintaining the pH in the range of ˜5.8-6.5 (50 mM succinic acid, pH 6.0), and containing sucrose (˜5% w/v).

Further contemplated in the above formulations is the presence of a stabilizing surfactant, in order to confer additional stability to the starting solutions of each product such that they may not then require storage under frozen or freeze-dried conditions.

These and further aspects of the invention will be apparent to those skilled in the art.

### DETAILED DESCRIPTION

A “stable” formulation is one in which the antibody or immunoconjugate (both herein referred also simply as protein), as the case may be, therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature and other storage conditions for a selected time period.

A protein “retains its physical stability” in a pharmaceutical formulation if it shows no signs of aggregation, precipitation and/or denaturation upon visual examination of color and/or clarity, or as measured by UV light scattering or by size exclusion chromatography.

A protein “retains its chemical stability” in a pharmaceutical formulation, if the chemical stability at a given time is such that the protein is considered to still retain its biological activity as defined below. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example.

An antibody “retains its biological activity” in a pharmaceutical formulation, if the biological activity of the antibody at a given time is within about 20% (within the errors of the assay) of the biological activity exhibited at the time the pharmaceutical formulation was prepared as determined in an antigen binding assay, for example.

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the

FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al, Nature 332:323-329 (1988); and Presta, Curr. Op. Struct. Biol. 2:593-596 (1992); U.S. Pat. No. 5,639,641.

The term “hypervariable region” when used herein refers to the amino acid residues of an antibody which are responsible for antigen-binding. The hypervariable region comprises amino acid residues from a “complementarity determining region” or “CDR” (e.g. principly residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. principly residues 26-32 (L1), 50-52 (L2) and 91-96 (L3) in the light chain variable domain and 26-32 (H1), 53-55 (H2) and 96-101 (H3) in the heavy chain variable domain; Chothia Lesk J. Mol. Biol. 196:901-917 (1987)). “Framework” or “FR” residues are those variable domain residues other than the hypervariable region residues as herein defined.

The humanized C242 has variable heavy and light chain amino acid sequences (SEQ ID NO: 1 and 2, respectively) as shown below.

SEQ ID NO:1

QVQLVQSGAEVKKPGETVKISCKASDYTFTYYGMNWVKQAPGQGLKWMGW
IDTTTGEPTYAQKFQGRIAFSLETSASTAYLQIKSLKSEDTATYFCARRG
PYNWYFDVWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK.

SEQ ID NO:2

DIVMTQSPLSVPVTPGEPVSISCRSSKSLLHSNGNTYLYWFLQRPGQSPQ
LLIYRMSNLVSGVPDRFSGSGSGTAFTLRISRVEAEDVGVYYCLQHLEYP
FTFGPGTKLELKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

Technologies in making huC242-DM1 are described in U.S. Pat. Nos 5,208,020; 5,552,293; 5,639,641; and EP528,527.

The antibody which is to be formulated is preferably essentially pure and desirably essentially homogeneous (i.e. free from contaminating proteins etc). “Essentially pure” antibody means a composition comprising at least about 90% by weight of the antibody, based on total weight of the composition, preferably at least about 95% by weight. “Essentially homogeneous” antibody means a composition comprising at least about 99% by weight of antibody, based on total weight of the composition.

The symbol “˜” means “about”.

huC242-DM1 to be formulated has not been subjected to prior lyophilization and the formulation of interest herein is an aqueous formulation. An aqueous formulation for huC242-DM1 is prepared comprising ˜1-30 mg/mL of huC242-DM1 in a pH-buffered solution. The buffer of this invention has a pH in the range from about 5.8 to about 6.2, preferably about pH 6.0. Examples of buffers that will control the pH within this range include acetate (e.g. sodium acetate), succinate (such as sodium succinate), gluconate, histidine, citrate and other organic acid buffers. The buffer concentration can be from about 1 mM to about 100 mM, preferably from about 50 mM. The preferred buffer is succinic acid (about 50 mM), pH 6.0.

A polyol, which acts as a tonicifier and may stabilize huC242-DM1, is included in the formulation. In preferred embodiments, the polyol is a nonreducing sugar, such as sucrose or trehalose. Preferred polyol is sucrose in about 5% w/v.

A surfactant can also be added to the huC242-DM1 formulation. Exemplary surfactants include nonionic surfactants such as polysorbates (e.g. polysorbates 20, 80 etc) or poloxamers (e.g. poloxamer 188). The amount of surfactant added is such that it reduces aggregation of the formulated immunoconjugate and/or minimizes the formation of particulates in the formulation and/or reduces adsorption. For example, the surfactant may be present in the formulation in an amount from about 0.001% to about 0.5%, preferably from about 0.005% to about 0.2% and most preferably from about 0.01% to about 0.1%. The addition of Pluronic F68, can also be concieved in case where a solution dosage form was desired.

The stabilizing formulation for antibody C242 is prepared comprising ˜1-30 mg/mL of C242 in a pH-buffered solution. The buffer of this invention has a pH in the range from about 5.8 to about 6.5, preferably about pH 6.0. Examples of buffers that will control the pH within this range include acetate (e.g. sodium acetate), succinate (such as sodium succinate), gluconate, histidine, citrate and other organic acid buffers. The buffer concentration can be from about 1 mM to about 100 mM, preferably about 50 mM, depending, for example, on the buffer. The preferred buffer is succinic acid (about 50 mM), pH 6.0. An polyol, which acts as a tonicifier and may stabilize C242, is included in the formulation. In preferred embodiments, the polyol is a nonreducing sugar, such as sucrose or trehalose. Preferred polyol is sucrose in about 5% w/v. Preferably the formulation will stabilize C242 for 2 years or longer under −70° C. frozen storage during the interim between initial antibody manufacture and conjugation to form huC242-DM1.

The invention will be more fully understood by reference to the following examples. They should not, however, be construed as limiting the scope of the invention. All literature and patent citations are incorporated herein by reference.

### Specific Embodiments

A variety of challenging stability problems were encountered during the development of a novel therapeutic monoclonal antibody (mAb) C242 (immunoconjugate) and its immunoconjugate huC242-DM1. These challenges were related primarily to degradation in the form of aggregation (soluble and insoluble) of the protein while in solution, and were resolved via formulation studies and dosage form design. Pre-formulation studies were designed to identify the appropriate pH environment for the stability of the mAb with a minimum of additional formulation excipients. Inclusion of surfactants was examined in order to assess any effects on stability. Sucrose served as a bulking agent, as well as, a cryoproctectant for lyophilization cycle development. Prospective solution formulations were tested in order to determine sensitivities to freeze/thaw cycling, vigorous shaking, stress storage, and light. The protein formulations were subjected to a battery of analyses to assure the potency, purity, and quality of the material, which included, among others pH, appearance, UV/VIS, SDS-PAGE, SEC, ELISA, Bioassay, and cIEF. A final formulation of 50-mM succinic acid, pH 6.0, containing 5.0% sucrose was shown to confer a sufficiently stable environment for a lyophilized immunoconjugate dosage form. However, it was determined that, the addition of a surfactant, such as Pluronic F68, should be considered in the case where a solution dosage form was desired.

What is claimed is:

1. A stable aqueous formulation of huC242-DM1 suitable for subsequent lyophilization comprising huC242-DM1 in the concentration range of about 1 to 20 mg/mL, in a buffer maintained at pH in the range of about 5.8 to 6.2, and sucrose in about 5% w/v.

2. The formulation of claim 1 in which pH is maintained at 6 with between 1 to 100 mM succinic acid.

3. The formulation of claim 2 in which the concentration of succinic acid is at 50 mM.

4. A stable frozen formulation for monoclonal antibody C242, comprised of C242 in the concentration range of about 1 to 30 mg/mL in a buffer maintained at pH in the range of about 5.8 to 6.5, and sucrose in about 5% w/v.

5. The formulation of claim 4 in which pH is maintained at 6 with between 1 to 100 mM succinic acid.

6. The formulation of claim 5 in which the concentration of succinic acid is at 50 mM.


SEQUENCE LISTING (2 sequences)

SEQ ID NO: 1 - protein, 449 aa, human
QVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, human
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC